(19) United States
(12) Patent Application Publication
(10) Pub. No.: US 2021/0403230 A1
(43) Pub. Date: Dec. 30, 2021

(54) SOLID ORGANIC PEROXIDE COMPOSITION

(71) Applicant: NOURYON CHEMICALS INTERNATIONAL B.V., ARNHEM (NL)

(72) Inventors: Wilhelm Klaas FRIJLINK, Zwolle (NL); Petrus Paulus WAANDERS, Goor (NL); Johan NUYSINK, Rijssen (NL); Rudy KOERS, Deventer (NL)

(73) Assignee: NOURYON CHEMICALS INTERNATIONAL B.V., ARNHEM (NL)

(21) Appl. No.: 17/284,061

(22) PCT Filed: Oct. 11, 2019
(86) PCT No.: PCT/EP2019/077671

§ 371 Date: Apr. 9, 2021

(30) Foreign Application Priority Data
Oct. 12, 2018 (EP) 18200136.2

(51) Int. Cl. B65D 85/84; C08J 7/12; C09K 15/06; C09K 15/02; B01F 3/18; B01F 9/00

(52) U.S. Cl. CPC B65D 85/84; C08J 7/12; C09K 15/06; B01F 2215/0036; B01F 3/18; B01F 9/00; C08J 2323/12; C09K 15/02

(57) ABSTRACT

Organic peroxide composition that is solid at room temperature, said composition comprising (i) at least about 40 wt %—based on the weight of the entire composition—of an organic peroxide that is solid at room temperature, said organic peroxide being selected from peroxydicarbonates and diacylperoxides, and (ii) about 0.001 to about 5 wt %—based on the weight of organic peroxide in the composition—of an HCl scavenger that is solid at room temperature.

### CROSS-REFERENCE TO RELATED APPLICATION

This application is a U.S. National-Stage entry under 35 U.S.C. § 371 based on International Application No. PCT/EP2019/077671, filed Oct. 11, 2019 which was published under PCT Article 21(2) and which claims priority to European Application No. 18200136.2, filed Oct. 12, 2018, which are all hereby incorporated in their entirety by reference.

### TECHNICAL FIELD

The present disclosure relates to a solid organic peroxide composition that can be suitably stored and transported in stainless steel containers.

### BACKGROUND

Liquid organic peroxide compositions, generally containing organic peroxide diluted with an organic solvent or suspended or emulsified in water, are conventionally stored and transported in HDPE-based containers or stainless steel containers. Corrosion of the so-filled stainless steel containers is not an issue.

Solid organic peroxides are conventionally stored and transported in HDPE-based containers or LDPE-based bags. However, handling of small LDPE bags is labor intensive and may cause dusting, with consequential safety issues, whereas transportation of solid peroxydicarbonates and diacyl peroxides in big bags (super sacks or flexible IBCs) is not allowed by UN regulations. Furthermore, safety requirements on industrial plants and factories may require a container to have an electrical resistivity that can only be obtained with stainless steel containers. In addition, the durability of HDPE containers is limited to about 5 years.

In storing and transporting certain solid organic peroxides in stainless steel containers, it has turned out that the containers tend to corrode. This corrosion not only limits the lifetime of the container, but also releases iron ions (and potentially other metal ions) in the peroxide. Transition metals, such as iron, are known to catalyse peroxide decomposition, which is evidently undesired.

### BRIEF SUMMARY

This disclosure provides an organic peroxide composition that is solid at room temperature, the composition comprising:

at least about 40 wt %—based on the weight of the entire composition—of an organic peroxide that is solid at room temperature, said organic peroxide chosen from peroxydicarbonates and diacylperoxides, and

from about 0.001 to about 5 wt %—based on the weight of organic peroxide in the composition—of an HCl scavenger that is solid at room temperature.

This disclosure also provides a stainless steel container comprising:
a solid composition comprising at least about 40 wt %—based on the weight of said solid composition—of an organic peroxide that is solid at room temperature and is selected from peroxydicarbonates and diacylperoxides,
an HCl scavenger that is solid at room temperature, in an amount of at least about 0.001 wt %, based on the weight of organic peroxide that is present in the stainless steel container.

This disclosure further provides a method for storing and/or transporting an organic peroxide composition that is solid at room temperature in a stainless steel container, said method involving the addition to a stainless steel container of:

(i) a solid composition comprising at least about 40 wt %—based on the weight of said solid composition—of an organic peroxide that is solid at room temperature and is selected from peroxydicarbonates and diacylperoxides, and

(ii) at least about 0.001 wt %, based on the weight of organic peroxide that is present in the stainless steel container, of an HCl scavenger that is solid at room temperature.

### DETAILED DESCRIPTION

The following detailed description is merely exemplary in nature and is not intended to limit the present disclosure or the application and uses of the present disclosure. Furthermore, there is no intention to be bound by any theory presented in the preceding background of the present disclosure or the following detailed description.

It is therefore an object of the present disclosure to provide a solid organic peroxide composition that can be suitably stored and transported in stainless steel containers, without corrosion of the container.

Investigations towards the cause of the corrosion have resulted in the conclusion that it is the residual chloroformate or acid chloride in the organic peroxide which, in combination with moisture or residual water in the product, leads to the formation of HCl fumes. These HCl fumes lead to corrosion of the stainless steel container.

Solid organic peroxides that are prepared from chloroformates are peroxydicarbonates. Peroxydicarbonates are conventionally prepared by reacting a chloroformate with hydrogen peroxide in alkaline medium.

Solid organic peroxides that are prepared from acid chlorides are diacyl peroxides. Diacyl peroxides are conventionally prepared by reacting an acid chloride with hydrogen peroxide in alkaline medium.

Upon transport and storage of these solid organic peroxides in stainless steel containers, the HCl fumes tend to corrode the stainless steel in the presence of oxygen and moisture.

It has now been found that solid peroxydicarbonate and diacylperoxide compositions that contain a small amount of an HCl scavenger can be safely stored and transported in stainless steel containers without leading to corrosion.

At the same time, these compositions are safe and stable, meaning that the HCl scavenger does not promote the decomposition of the organic peroxide, can be mixed with the organic peroxide without significant segregation, and remains flowable.

The present disclosure therefore relates to an organic peroxide composition comprising:
at least about 40 wt %—based on the weight of the entire composition—of an organic peroxide that is solid at room temperature, said organic peroxide being selected from peroxydicarbonates and diacylperoxides,
about 0.001-5 wt %—based on the weight of organic peroxide in the composition—of an HCl scavenger that is solid at room temperature.

The present disclosure also relates to a packaged organic peroxide composition comprising said organic peroxide composition in a stainless steel container.

Suitable solid HCl scavengers include
metal carboxylates, which include the metal salts of mono-, di- and tri-carboxylic acids. Examples of metal salts of mono-carboxylic acids are metal stearates, metal lactates, and metal lactylates, such as Ca, Mg, Zn, Al, Na, K, and Li stearates, lactates, or lactylates; more preferably calcium stearate and calcium stearoyl-2-lactylate. Examples of metal salts of dicarboxylic acids are metal hexahydrophthalates or metal bicyclic[2.2.1]heptane dicarboxylates, such as Ca, Mg, Zn, Al, Na, K, and Li hexahydrophthalates or bicyclic[2.2.1]heptane dicarboxylates. Examples of metal salts of tricarboxylic acids are metal citrates, such as Ca, Mg, Zn, Al, Na, K, and Li mono-, di-, and tricitrates, more in particular sodium monocitrate or potassium monocitrate.
metal carbonates and metal bicarbonates, such as Ca, Mg, Zn, Na, K, or Li carbonate, or Na, K, or Li bicarbonate. A preferred (bi)carbonate is calcium carbonate;
metal oxides, such as CaO, MgO, and ZnO, with CaO being the preferred metal oxide;
metal silicates, such as calcium silicate or magnesium silicate
anionic clays;
and combinations thereof.

Anionic clays—also called hydrotalcites or layered double hydroxides—have a crystal structure including positively charged layers built up of specific combinations of divalent and trivalent metal hydroxides between which there are anions and water molecules. Hydrotalcite is an example of a naturally occurring anionic clay, in which carbonate is the predominant anion and the layers contain Mg and Al. Various natural, synthetic, and modified forms with other di- and/or trivalent metals and/or anions (including nitrate anions, organic anions and pillaring anions) are known. The anionic clay should be able to exchange the anions in its interlayer for chloride anions. Preferred anionic clays are anionic clays with carbonate or hydroxide anions in the interlayers.

The metal carboxylates, metal oxides, metal silicates, and metal (bi)carbonates neutralize the HCl. The anionic clays exchange anions (e.g. carbonate) for chloride ions, thereby encapsulating the chloride ions in the clay structure.

Suitable solid peroxydicarbonates are dicetyl peroxydicarbonate, dimyristyl peroxydicarbonate, dicyclohexyl peroxydicarbonate, and di(tert-butylcyclohexyl) peroxydicarbonate.

Suitable solid diacylperoxides include aliphatic and aromatic diacylperoxides. Preferred aliphatic diacylperoxides are those with aliphatic chains of at least 10 carbon atoms, such as dilauroyl peroxide.

Preferred aromatic diacylperoxides include dibenzoyl peroxide and substituted dibenzoyl peroxides, such as di(2-methylbenzoyl)peroxide, di(4-methylbenzoyl)peroxide, and di(2,4-dichlorobenzoyl)peroxide.

Peroxydicarbonates and diacylperoxides find use in processes for enhancing the melt strength of polypropylene (towards high melt strength polypropylene; HMS-PP) by extruding said polypropylene in the presence of the peroxydicarbonate or diacylperoxide. The present disclosure therefore also relates to the use of the composition according to the present disclosure for the modification of polypropylene, such as the production of HMS-PP.

Acid scavengers like calcium stearate, calcium stearoyl-2-lactylate, calcium lactate, and/or anionic clays are already used as additives in a polypropylene (modification) process for the purpose of trapping acidic catalyst residues that may deteriorate the polypropylene. This means that the introduction of calcium stearate, calcium stearoyl-2-lactylate, calcium lactate, and/or anionic clays, but also calcium carbonate (the carbonate is released as CO2 during polypropylene modification) via the composition according to the present disclosure, does not introduce any new materials in the modified polypropylene. Therefore, calcium stearate, calcium stearoyl-2-lactylate, calcium lactate, anionic clays, and/or calcium carbonate are preferred HCl scavengers in the composition of the present disclosure.

Peroxydicarbonates specifically suitable for this polypropylene modification are dicetyl peroxydicarbonate, dimyristyl peroxydicarbonate, and di(tert-butylcyclohexyl) peroxydicarbonate. A diacyl peroxides specifically suitable for this polypropylene modification is di(4-methylbenzoyl)peroxide. Therefore, in a preferred embodiment, the composition according to the present disclosure contains one or more of these peroxydicarbonates and diacyl peroxides.

Even more preferred are compositions containing a peroxydicarbonate or diacyl peroxide selected from the group including dicetyl peroxydicarbonate, dimyristyl peroxydicarbonate, di(tert-butylcyclohexyl) peroxydicarbonate, and di(4-methylbenzoyl)peroxide in combination with an HCl scavenger selected from the group including calcium stearate, calcium stearoyl-2-lactylate, anionic clay, and/or calcium carbonate.

The most preferred organic peroxides are dicetyl peroxydicarbonate and di(4-methylbenzoyl)peroxide.

The organic peroxide composition according to the present disclosure comprises at least about 40 wt %, preferably at least about 50 wt %, more preferably at least about 60 wt %, even more preferably at least about 85 wt %, and most preferably at least about 90 wt % —based on the weight of the entire composition—of an organic peroxide that is solid at room temperature, said organic peroxide being selected from peroxydicarbonates and diacylperoxides.

The organic peroxide composition according to the present disclosure comprises about 0.001 to about 5 wt %, preferably about 0.005 to about 2.5 wt %, and most preferably about 0.01 to about 0.5 wt %—based on the weight of organic peroxide in the composition—of an HCl scavenger that is solid at room temperature.

Other components that can be present in the organic peroxide composition are water, phlegmatizers or diluents, or additives, provided that the organic peroxide composition remains a solid material.

The composition according to the present disclosure can have the form of a physical mixture of organic peroxide particles and HCl scavenger particles.

Alternatively, it has the form of particles (powder, flakes, granules, pills, pellets, or other solid particle form) containing both the peroxydicarbonate and HCl scavenger in one particle.

The organic peroxide composition according to the present disclosure can be prepared in several ways.

In one embodiment, the organic peroxide and the HCl scavenger are physically mixed. This mixing can be performed before addition of the composition to the stainless steel container for storage and transport. Alternatively, the organic peroxide and the HCl scavenger can be added to the stainless steel container individually, after which the ingredients are mixed inside the container.

Suitable mixing equipment includes tumble mixers and rotating screw mixers (e.g. Nauta mixers). Alternatively, the solids can be dosed by in-line mixing, e.g. by dosing to a rotating screw mixer or a conveying screw, under continuous mixing.

In a second embodiment, the HCl scavenger is added to the reactor in which the organic peroxide is produced. According to this embodiment, the HCl scavenger can be present during the reaction of the chloroformate or acid chloride with hydrogen peroxide in alkaline medium, or can be added to the reactor after the reaction has been completed.

In a third embodiment, the HCl scavenger is added to molten organic peroxide. The resulting product can then be transformed into solid particles by, e.g. extrusion or granulation.

The present disclosure also relates to a packaged organic peroxide composition comprising the organic peroxide composition as described above in a stainless steel container.

The present disclosure also relates to a stainless steel container comprising:
a solid composition comprising at least about 40 wt %, preferably at least about 50 wt %, more preferably at least about 60 wt %, even more preferably at least about 85 wt %, and most preferably at least about 90 wt %—based on the weight of the entire solid composition—of an organic peroxide that is solid at room temperature and is selected from peroxydicarbonates and diacylperoxides,
an HCl scavenger that is solid at room temperature, in an amount of at least about 0.001 wt %, preferably about 0.001 to about 5 wt %, more preferably about 0.005 to about 2.5 wt %, and most preferably about 0.01 to about 0.5 wt %—based on the weight of organic peroxide present in the container.

According to one embodiment, the HCl scavenger and the solid composition can be admixed according to one of the preparation embodiments described above in order to form an organic peroxide composition according to the present disclosure. In another embodiment, the HCl scavenger is not admixed with the solid composition, but is present in a separate compartment within the container. Said separate compartment should be permeable to HCl fumes.

As explained above, the filled stainless steel container will not be subject to corrosion by its contents. The term “container” refers to any type of packaging that can be closed and can be used to store and transport solid organic peroxide compositions.

The container preferably has a size of about 200 to about 4000 liters, more preferably about 500-about 1500 liters. This includes stainless steel intermediate bulk containers (IBC's). A specifically preferred type of container is a Cone Valve IBC, which is specifically suited for powders as it prevents typical powder flow problems such as bridging, blockages, segregation, flushing, and core-flow. Such containers are available from Matcon®.

As mentioned above, the organic peroxide composition according to the present disclosure finds use in the modification of polypropylene, in particular the production of high melt strength polypropylene (HMS-PP). In this modification, the composition is added to the polypropylene prior to or during extrusion. The composition may be added to the polypropylene after suspending it in water, dispersing it in an inert solvent such as isododecane, or in any solid physical form, e.g. flakes or powder. The quantity of organic peroxide to be used will depend on the desired degree of modification and on the type of polypropylene employed. Preferably, use is made of organic peroxide concentrations in the range of about 0.1 to about 3.0 g of peroxide per about 100 g polypropylene, more preferably in the range of about 0.5 to about 2.0 g per about 100 g polypropylene; all calculated as pure and dry organic peroxide.

### EXAMPLES

### Example 1

A composition was prepared by tumble mixing a HDPE flask containing dicetyl peroxydicarbonate flakes (containing 97.5 wt % peroxide and 0.3 wt % cetylchloroformate) with different amounts (in wt % based on the organic peroxide weight) of different acid scavengers.

Experiments with an additional amount of cetylchloroformate (0.9 wt %) were also performed.

The following acid scavengers were used: calcium stearate, hydrotalcite (DHT-4A, ex-Kisuma), calcium carbonate.

Pre-cleaned smooth stainless steel (SS) 316L coupons (20×10×1 mm) were weighed and subsequently placed in a glass vial with approximately 1 g organic peroxide composition.

The open glass vial was placed into a 250 ml HDPE flask containing approximately 20 g organic peroxide composition. The HDPE flask was closed with a screw-cap and was stored for at least 12 days at 20° C.

After the storage period, the coupons were visually inspected for corrosion and, after the below cleaning procedure, re-weighed to determine the weight loss. According to the cleaning procedure, any rust was removed from the corroded coupons with a wetted sponge (water/abrasive crème). The coupons were rinsed with distilled water and then with acetone, dried in an oven at 60° C. for 1 hour, and cooled to room temperature.

TABLE 1

| | | | | | | |
|---|---|---|---|---|---|---|
| | HCl | Stainless | days at | | Weight | |
| Exp. | scavenger | steel type | 20° C. | Corrosion? | loss (%) | Observations |

No acid scavenger

| | | | | | | |
|---|---|---|---|---|---|---|
| 1 | — | 316L | 12 | ++ | 0.108 | clear corrosion, |
| | | | | | | brown droplets |
| | | | | | | and deterioration |
| | | | | | | of coupon |
| 2 | — | 254 | 12 | + | 0.027 | Corrosion, but |
| | | SMO | | | | less than 316L |

Ca-stearate

| | | | | | |
|---|---|---|---|---|---|
| 3 | 0.05% wt % | 316L | 14 | −− | no corrosion |
| | Ca-stearate | | | | |
| 4 | 0.25% wt % | 316L | 60 | −− | no corrosion |
| | Ca-stearate | | | | |

DHT-4A

| | | | | | |
|---|---|---|---|---|---|
| 5 | 0.01 wt % | 316L | 19 | −− | no corrosion |
| | DHT-4A | | | | |
| 6 | 0.025 wt % | 316L | 42 | −− | no corrosion |
| | DHT-4A | | | | |
| 7 | 0.2 wt % | 316L | 19 | −− | no corrosion |
| | DHT-4A | | | | |

CaCO3

| | | | | | |
|---|---|---|---|---|---|
| 8 | 0.016 wt % | 316L | 19 | −− | no corrosion |
| | CaCO3 | | | | |
| 9 | 0.04 wt % | 316L | 19 | −− | no corrosion |
| | CaCO3 | | | | |
| 10 | 0.1 wt % | 316L | 19 | −− | no corrosion |
| | CaCO3 | | | | |

Dicetyl peroxydicarbonate flakes with additional 0.9 wt % cetylchloroformate

| | | | | | | |
|---|---|---|---|---|---|---|
| 11 | — | 316L | 14 | ++ | 0.220 | clear corrosion |
| 12 | 2.5 wt % | 316L | 19 | −− | | no corrosion |
| | Ca stearate | | | | | |

Influence of temperature on corrosion; no HCl scavenger

| | | | | | | |
|---|---|---|---|---|---|---|
| 13 | 10° C. | 316L | 12 | ++ | n.d. | clear corrosion |
| 14 | 20° C. | 316L | 12 | ++ | n.d. | clear corrosion |

The results show that the organic peroxide composition, without HCl scavenger, corrodes 316L stainless steel. The results also show that it corrodes a higher quality stainless steel 254 SMO; a stainless steel that is resistant to dilute HCl solutions.

### Example 2

Several compositions prepared in Example 1 were tested for their corroding effects using the following test set-up:

Pre-cleaned smooth 316L stainless steel coupons (20×10×1 mm) were weighed and subsequently placed in a 250 ml HDPE flask: directly on top, or below a 21 g sample of the dicetyl peroxydicarbonate-containing composition.
The HDPE flask was closed with a screw-cap and was stored for at least 11 days at 20° C. After the test, the coupons were inspected, cleaned and weighed as explained in Example 1.

TABLE 2

| | | | | | |
|---|---|---|---|---|---|
| | | | | Weight | |
| Exp. | HCl scavenger | days at 20° C. | Corrosion? | loss (%) | Observations |

Coupon below the composition

| | | | | | |
|---|---|---|---|---|---|
| 15 | — | 13 | ++ | 0.097 | Clear |
| | | | | | corrosion |
| 16 | 0.05 wt % Ca stearate | 11 | −− | | No corrosion |
| 17 | 0.1 wt % Ca stearate | 11 | −− | | No corrosion |
| 18 | 0.5 wt % Ca stearate | 13 | −− | | No corrosion |

Coupon on top of the composition

| | | | | | |
|---|---|---|---|---|---|
| 19 | — | 13 | ++ | 0.187 | Clear |
| | | | | | corrosion |
| 20 | 0.05 wt % Ca stearate | 11 | −− | | No corrosion |
| 21 | 0.1 wt % Ca stearate | 11 | −− | | No corrosion |

These results again confirm that the HCl scavenger is able to prevent corrosion of all inner walls of a stainless steel container.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Organic peroxide composition that is solid at room temperature, said composition comprising:
at least about 40 wt %—based on the weight of the entire composition—of an organic peroxide that is solid at room temperature, said organic peroxide chosen from peroxydicarbonates and diacylperoxides,
from about 0.001 to about −5 wt %—based on the weight of organic peroxide in the composition—of an HCl scavenger that is solid at room temperature.

2. Organic peroxide composition according to claim 1 wherein the composition comprises from about 0.005 to about −2.5 wt %, based on the weight of said organic peroxide in the composition—of said HCl scavenger.

3. Organic peroxide composition according to claim 1 wherein the composition comprises at least about 85 wt %, based on the weight of the entire composition—of said organic peroxide.

4. Organic peroxide composition according to claim 1 wherein the organic peroxide is a peroxydicarbonate chosen from dicetyl peroxydicarbonate, dimyristyl peroxydicarbonate, dicyclohexyl peroxydicarbonate, and di(tert-butylcyclohexyl) peroxydicarbonate.

5. Organic peroxide composition according to claim 1 wherein the organic peroxide is a diacylperoxide chosen from aliphatic diacylperoxides with aliphatic chains of at least about 10 carbon atoms, dibenzoyl peroxide, and substituted dibenzoyl peroxides.

6. Organic peroxide composition according to claim 1 wherein the HCl scavenger is chosen from metal carboxylates, metal oxides, metal (bi)carbonates, metal silicates, anionic clays, and combinations thereof.

7. Organic peroxide composition according to claim 6 wherein the HCl scavenger is chosen from calcium stearate, calcium stearoyl-2-lactylate, CaO, calcium carbonate, and anionic clays.

8. Packaged organic peroxide composition comprising the organic peroxide composition according to claim 1 in a stainless steel container.

9. Stainless steel container comprising the organic peroxide composition according to claim 1.

10. Stainless steel container comprising:
a solid composition comprising at least about 40 wt %—based on the weight of said solid composition—of an organic peroxide that is solid at room temperature and is selected from peroxydicarbonates and diacylperoxides,
an HCl scavenger that is solid at room temperature, in an amount of at least about 0.001 wt %, based on the weight of organic peroxide that is present in the stainless steel container.

11. Method for storing and/or transporting an organic peroxide composition that is solid at room temperature in a stainless steel container, said method involving the addition to a stainless steel container of:
(i) a solid composition comprising at least about 40 wt %—based on the weight of said solid composition—of an organic peroxide that is solid at room temperature and is selected from peroxydicarbonates and diacylperoxides, and
(ii) at least about 0.001 wt %, based on the weight of organic peroxide that is present in the stainless steel container, of an HCl scavenger that is solid at room temperature.

12. Process for producing the organic peroxide composition according to claim 1, comprising the step of physically mixing said organic peroxide and said HCl scavenger.

13. Process for producing the organic peroxide composition according to claim 1, wherein said HCl scavenger is added to the reactor in which the organic peroxide is produced.

14. Process for producing the organic peroxide composition according to claim 1, comprising the step of adding said HCl scavenger to molten organic peroxide.

15. (canceled)

16. Organic peroxide composition according to claim 1 wherein the composition comprises from about 0.01 to about 0.5 wt %—based on the weight of said organic peroxide in the composition—of said HCl scavenger.

17. Organic peroxide composition according to claim 1 wherein the composition comprises at least about 90 wt %, based on the weight of the entire composition—of said organic peroxide.

18. Organic peroxide composition according to claim 6 wherein the HCl scavenger is chosen from hydrotalcite, calcium stearate, and calcium carbonate.

19. Organic peroxide composition according to claim 1 wherein
the composition comprises from about 0.01 to about 0.5 wt %—based on the weight of said organic peroxide in the composition—of said HCl scavenger;
the composition comprises at least about 90 wt %, based on the weight of the entire composition—of said organic peroxide; and
wherein the HCl scavenger is chosen from hydrotalcite, calcium stearate, and calcium carbonate.
